(19) United States
(12) Patent Application Publication
(10) Pub. No.: US 2024/0077442 A1
(43) Pub. Date: Mar. 7, 2024

(54) SENSOR DEVICE AND MANUFACTURING METHOD THEREFORE

(71) Applicant: Power Roll Limited, Sunderland Tyne and Wear (GB)

(72) Inventors: Fabrice Dierre, Sunderland Tyne and Wear (GB); Alexander John Topping, Sunderland Tyne and Wear (GB); Nicholas Kasch, Sunderland Tyne and Wear (GB)

(21) Appl. No.: 18/268,483

(22) PCT Filed: Dec. 20, 2021
(86) PCT No.: PCT/GB2021/053370

§ 371 Date: Jun. 20, 2023

(30) Foreign Application Priority Data
Dec. 23, 2020 (GB) 2020563.9

(51) Int. Cl. G01N 27/12

(52) U.S. Cl. CPC G01N 27/12

(57) ABSTRACT

The present invention relates to a sensor device, in particular the invention relates to an electronic sensor device formed within a groove of a substrate adapted such that in response to engagement with an analyte a signal response is provided. The invention also relates to a method of forming such a sensor device. The electronic sensor device comprises a substrate comprising at least one groove, said groove including a first face and a second face, said groove having a cross-sectional profile including at least a groove depth In within the substrate and a groove width at a surface of said substrate. Said first face includes a first electrically non-insulating portion and said second face including a second electrically non-insulating portion wherein, within said profile, said first electrically non-insulating portion is electrically separated from said second electrically non-insulating portion. A detection medium is provided within said groove, arranged to contactingly engage said first and second electrically non- insulating portions, and adapted to be contactingly engaged by an analyte. Furthermore, said profile or said detection medium is adapted such that, in use, a signal response is provided comprising a first response phase and a subsequent second response phase in response to an engagement of the analyte with the detection medium.

The present invention relates to a sensor device, in particular the invention relates to an electronic sensor device formed within a groove of a substrate adapted such that in response to engagement with an analyte a signal response is provided. The invention also relates to a method of forming such a sensor device.

### BACKGROUND

Sensor devices which provide an electrical signal in response to analytes in their environment are well known. For example, certain sensors provide a measurable change in capacitance in response to an analyte, or a chemical group of analytes, due to the specific response of capacitor dielectric material to the analyte in question. In this way, the dielectric material of such devices is chosen because of its affinity for the target analyte or analytes in question. Known examples include lanthanide fluoride to detect adsorption of gases; polyimide to detect ionic species in a liquid; and palladium to detect heavy metals.

In this way, the form and nature of the sensor is dependent upon a target analyte or a target chemical group of analytes. That is, the sensor is specifically constructed and adapted in order to ensure an increase or decrease of the electrical capacitance of a device according to a particular environment and the analyte that the sensor detects.

Certain electronic sensor devices which rely on a capacitance signal utilise traditional capacitor materials and arrangements, for example, using crystalline wafers sandwiched between electrodes, or inclusion of multi-layered structures including metal plate electrodes.

Certain other capacitance electronic sensor devices rely on accurate spacing between electrodes in order to create a device capable of providing an alternative capacitance signal in response to contact with the electrodes by the target analyte or analytes.

Known examples include palladium-coated silver wires mounted onto polyester substrate; silicone-rubber coated metallic fingers mounted to a glass or alumina substrate; and a MEMS device with planar electrodes accurately positioned apart using supporting spacer.

A common drawback of the solutions according to the prior art is that devices are limited to respond to specific analytes, within specific environments. A further drawback is that the devices are expensive to produce due to their complexity and requirement for specialist materials, or accurate spacing of components.

Furthermore, the capacitance signal response of known devices may be susceptible to long term degradation, for example due to the gradual modification of the dielectric over time. In this way, known devices may provide a capacitance change which is undetectable because it occurs slowly over a prolonged time period.

Additionally, sensor devices for certain analytes may require pre-treatment before use. In particular, known sensors for example hydrogen gas typically require heating or thermal treatment for a number of hours, perhaps 24 hours or more, in order to ensure the sensor is decontaminated so as to ensure accurate and timely response to the analyte. Thus, a sensor requires coupling to a high-power electrical supply and requires significant energy and time for setting up before being ready to use.

Accordingly, it is an object of the invention to alleviate one or more of the above drawbacks. In particular, it is an object of the invention to provide an electronic sensor capable of providing an electrical signal in response to a broad range of analytes. That is, the electronic sensor is readily adaptable to provide an electrical signal for different analytes and different chemical groups of analytes.

A further object of the invention is to provide a sensor device which provides a reliable electrical signal in response to an analyte. In other words, the device provides an electrical signal that is readily detectable and stable such that the presence of an analyte is quickly and definitively indicated.

A yet further object of the invention is to provide a sensor device and a method of manufacture which is a relatively simple design, so that the device may be manufactured quickly and at low cost. Another object is that the design and manufacturing method is easily modified in order to adapt the sensor in order to detect a range of analytes.

A further object of the invention is to provide a sensor device which is easy to install, and which is ready for immediate use after installation. That is, to provide a sensor device which can be used as soon as it is installed. It is also an object of the invention to provide a sensor device which operates using reduced electrical power.

### SUMMARY OF THE INVENTION

According to a first aspect of the invention, there is provided an electronic sensor device including:
a substrate including at least one groove, said groove including a first face and a second face, said groove having a cross-sectional profile including at least a groove depth within the substrate and a groove width at a surface of said substrate and;
said first face including a first electrically non-insulating portion and said second face including a second electrically non-insulating portion wherein, within said profile, said first electrically non-insulating portion is electrically separated from said second electrically non-insulating portion;
a detection medium provided within said groove, arranged to contactingly engage said first and second electrically non-insulating portions, and adapted to be contactingly engaged by an analyte; and
wherein said profile or said detection medium is adapted such that, in use, a signal response is provided including a first response phase and a subsequent second response phase in response to an engagement of the analyte with the detection medium.

The signal response includes first and second response phases. That is the signal response provides a characteristic first response phase and a subsequent, different second response phase. Without being bound to theory, the inventors believe the first and second response phases may be provided by two characteristic interactions provided by the engagement of the analyte with the detection medium. In particular, due to the contacting engagement of the detection medium with the two non-insulating portions of the groove, the detection medium provides a dielectric between two capacitive surfaces. Engagement of the detection medium by an analyte thereby changes or affects the dielectric characteristics of the detection medium as explained herein.

In certain examples, due to the contacting engagement of the detection medium with the two non-insulating portions of the groove, the detection medium provides a resistive element, that is an electrical resistor, between two non-insulating surfaces. Engagement of the detection medium by an analyte thereby changes or affects the resistance characteristics of the detection medium as explained herein.

As used herein, a detection medium may be a suitable material able to provide a measurable electrical characteristic which provides a suitable first and second phase responses to the sensor device. The choice of detection medium may depend on the analyte that is being detected.

As used herein electrically non-insulating portions may be electrical conductors or may act as a semiconductor. In certain examples, one or both of the first electrically non-insulating portion and the second electrically non-insulating portion may be electrical conductors. In certain examples, one or both of the first electrically non-insulating portion and the second electrically non-insulating portion may act as a semiconductor.

In certain examples, a first electrically non-insulating portion may be an electrical conductor and the second electrically non-insulating portion may act as a semiconductor.

The analyte may be a liquid. Where the analyte is a liquid, it may be that the detection medium is a gas, e.g. air. In this context, the engagement between the analyte and the detection medium may be the displacement of the gas (e.g. air) by the analyte. In these embodiments, the liquid will typically be non-insulating, e.g. a polar protic solvent. In certain examples the liquid may comprise water. In certain examples the liquid may be water or an aqueous solution. Where the liquid is water, the detection medium may be a gas, e.g. air. Where the liquid is a non-corrosive aqueous solution, the detection medium may be a gas, e.g. air.

Where the analyte is a corrosive aqueous solution, it may be that the faces of the groove are coated. In certain examples, the coating may be unreactive. ‘Unreactive’ is intended to mean that the coating does not react with the corrosive aqueous solution that is being detected. Optionally, the coating may completely coat the electrically non-insulating portion of a groove face.

The coating will typically be a dielectric. It may have a dielectric constant of 1 or greater. It may have a dielectric constant of 10 or greater. It may have a dielectric constant of 1000 or less, e.g. 100 or less.

The coating may absorb or adsorb at least a portion of the corrosive aqueous solution.

It may be that the coating swells when in contact with the corrosive aqueous solution, e.g. as a result of absorption or adsorption of the corrosive aqueous solution into the coating.

It may be that the dielectric constant of the coating changes when it is in contact with the corrosive aqueous solution, e.g. e.g. as a result of absorption or adsorption of the corrosive aqueous solution into the coating. The dielectric constate of coating may provide a swelling of the coating.

Where the coating does not swell or change its dielectric constant in contact with the analyte, it may be that the detection medium is a gas (e.g. air) and the engagement between the analyte and the detection medium is the displacement of the gas (e.g. air) by the analyte.

Where the coating does swell or change its dielectric constant, the detection medium may be the coating or the detection medium may be both the coating and a gas (e.g. air).

The coating may comprise a polymer. The coating may comprise an unreactive polymer. The coating may comprise a fluorinated polymer, e.g. polyvinyldifluoride (PVDF) or polytetrafluoroethylene (PTFE).

Where the corrosive aqueous solution is acidic (e.g. a solution comprising HCl, H₂SO₄, H₃PO₄, organic acids, etc.), illustrative polymers include PVDF and PTFE.

Where the corrosive aqueous solution is alkaline (e.g. a solution comprising hydroxide ions, carbonate ions, organic bases, etc.), illustrative polymers include PVDF, PTFE and poly(methyl methacrylate) (PMMA).

The coating may comprise additives to alter the properties of the polymer, e.g. to increase the dielectric constant of the polymer or to increase the propensity of the polymer to swell.

The coating may comprise a PVDF matrix and a barium titanate particulate filler. An illustrative example is sold under the tradename DuPont LuxPrint® 8153.

The coating may have a thickness in the range 10 nm to 10 μm. The coating thickness may vary, depending upon the groove width or the groove height. The coating may be formed as a single layer, or as multiple layers of a coating.

Where a coating is formed as multiple layers, each layer of the coating may be provided the different electrical conductivities. In this way, a first layer of a coating may be more electrically conductive or may be less electrically conductive than a second layer of the coating. One of, or a plurality of, the multiple layers of a coating may be an electrical conductor. Additionally, or alternatively, one of, or a plurality of, the multiple layers of a coating may act as a semiconductor.

The analyte may be a gas.

Where the analyte is a gas, typically the detection medium comprises a binder contactingly engaged with a first and second face of a groove, and an active component. The active component is selected to engage with the analyte and, through that engagement, alter the dielectric constant of the detection medium.

The binder will typically comprise a polymer. The binder may comprise a fluorinated polymer. The binder may be a printable polymer composition. Illustrative examples include polymer compositions comprising polystyrene, PVDF, PTFE and PMMA.

The binder may be a printable polymer composition comprising a fluorinated polymer. The binder may be a printable polymer composition comprising PVDF. An illustrative example is sold under the tradename DuPont LuxPrint® 8155.

The active component could be any substance that absorbs, adsorbs dissolves, intercalates, reacts with, or otherwise takes up the analyte.

The active component may be a liquid. Where the active component is a liquid, it may be present at such a concentration relative to the binder that the detection medium is a liquid. Alternatively, it may be present at such a concentration relative to the binder that the detection medium is a gel.

The active component may be a solid. Where the active component is a solid, it typically takes the form of a filler distributed through a matrix of the binder.

The active component may be non-insulating. Where the active component is a filler distributed through a binder, the active component may provide an electrically conductive pathway through the binder.

An active component may provide an electrically conductive pathway from the first face of the groove to the second face. The electrically conductive pathway may have a higher electrical resistance than an alternative conductive pathway from a groove face to an electric terminal provided outside of the groove.

The analyte may be CO₂ gas.

Where the analyte is CO₂, the active component may be an ionic liquid. An ionic liquid is an ionic compound that is liquid at 20° C. The ionic liquid may comprise an imidazolium ion. The imidazolium ion may comprise a hydrogen at the C₂-position. The ionic liquid may comprise a 1-alkyl-3-alkyl-imidazolium ion. The ionic liquid may comprise a 1-butyl-3-methyl imidazolium ion. The ionic liquid may comprise a 1-ethyl-30 3-methylimidazolium ion.

The counterion (e.g. the counterion that is associated with the imidazolium ion) of the ionic liquid may be a bis(alkylsulfonyl)imide or a bis(haloalkylsulfonyl)imide. The counterion may be bis(trifluoromethylsulfonyl)imide.

The ionic liquid may be 1-ethyl-3-methylimidazolium bis(trifluoromethylsulfonyl)imide.

The ionic liquid may also comprise an amine base, e.g. 1,8-Diazabicyclo[5.4.0]undec-7-ene (DBU). Typically, the amine base will be dissolved in the ionic liquid.

An illustrative example of a suitable ionic liquid comprises a 1-butyl-3-methyl imidazolium cation and DBU, e.g. 1-butyl-3-methyl imidazolium chloride and DBU.

The detection medium may comprise from 1% to 50% by weight ionic liquid relative to the total weight of the detection medium. The detection medium may comprise no more than 40% by weight ionic liquid relative to the total weight of the detection medium. The detection medium may comprise from 5% to 20% by weight ionic liquid relative to the total weight of the detection medium. The detection medium may comprise from 5% to 15% by weight ionic liquid relative to the total weight of the detection medium. When the ionic liquid is present at a concentration below about 15%-20% (depending on the identity of the ionic liquid and binder) by weight relative to the total weight of the detection medium, the detection medium is typically liquid.

When detecting CO₂, the detection medium may be a liquid.

A particularly suitable detection medium for CO₂ is a PVDF binder with 10% ionic liquid.

The analyte may be water vapour.

Where the analyte is water vapour, the active component may be an ionic liquid. The ionic liquid may be as described above for ionic liquids suitable for detecting CO₂.

The detection medium may comprise from 1% to 50% by weight ionic liquid relative to the total weight of the detection medium. The detection medium may comprise from 15% to 50% by weight ionic liquid relative to the total weight of the detection medium. The detection medium may comprise from 20% to 50% by weight ionic liquid relative to the total weight of the detection medium. When the ionic liquid is present at a concentration above about 15%-20% (depending on the identity of the ionic liquid and binder) by weight relative to the total weight of the detection medium, the detection medium is typically a gel.

When detecting water vapour, the detection medium may be a gel.

The analyte may be H₂ gas.

Where the analyte is H₂, the detection medium may be a binder including a filler.

The binder may include a fluorinated polymer. The binder may be a printable polymer composition.

The binder may be a printable polymer composition comprising a fluorinated polymer.

The binder may be a printable polymer composition comprising PVDF. The binder may be a printable polymer composition comprising polystyrene or comprising PMMA. An illustrative example is sold under the tradename DuPont LuxPrint® 8155.

Where the analyte is H₂, the active component may be a solid filler, e.g. a metal or metal oxide. The filler may be a substance, e.g. a metal or metal oxide, that binds molecular oxygen.

The filler, e.g. metal or metal oxide, may be distributed through a matrix of the binder. The filler may be in the form of nanostructures.

In particular embodiments, the active component may be zinc oxide. In other particular embodiments, the active component may be tin oxide. In further particular embodiments, the active component may be titanium oxide. The active component, such as zinc oxide or tin oxide, may be in the form of nanostructures. Nanostructures include nanowires, nanotubes, nanoparticles and nanofibers.

A catalyst may also be present in the detection medium. The catalyst may be a coating on the active component. The catalyst may comprise a transition metal, e.g. Pd. The catalyst may be a transition metal, e.g. Pd. The catalyst may be a transition metal compound, e.g. palladium chloride.

It may be that the active component is tin oxide and the catalyst comprises Pd. It may be that the active component is tin oxide and the catalyst is Pd. It may be that the active component is a tin oxide and the catalyst is palladium chloride. It may be that the active component is a tin oxide coated with palladium chloride.

The detection medium may comprise from 1% to 50% by weight filler (e.g. zinc oxide or tin oxide) relative to the total weight of the detection medium. Preferably, the detection medium may comprise from 2% to 10% by weight filler. Yet more preferably, the detection medium may comprise from 3% to 5% by weight filler.

If present, the detection medium may comprise from 0.01% to 5% by weight of the catalyst relative to the total weight of the detection medium.

Certain examples provide an electronic sensor device which does not require pre-treatment before use.

In particular, certain examples provide a device in which the analyte is hydrogen gas that is useable without pre-heating or thermal activation. That is a hydrogen sensor is provided which is ready for use as soon as it is installed in sensing apparatus. The sensor thus does not require heating for several hours or more so as to decontaminate the sensor as is typically the case with known sensors.

In the above discussion, where a substance or mixture is described as having a particular physical state (e.g. solid, liquid, gas, gel), that is the physical state of that substance or mixture at a temperature of 20° C. and a pressure of 1 atm.

In the first response phase, the signal response may be provided by a surface effect of the analyte upon the detection medium. That is, in the first response phase, the signal response may be provided by engagement with the analyte upon a surface of the detection medium. The first response phase thus provides a first change to the dielectric or electrical resistance characteristics of the sensor device formed within the groove.

The first change of the dielectric or electrical resistance characteristics may be suitably detected by measuring, for example, one or more of the capacitance, resistance or impedance across the device, as is set out in more detail herein. In the first response phase, a first rate of change is measured.

In the second response phase, the signal response may be provided by a bulk effect of the analyte upon the detection medium. That is, in the second response phase, the signal response may be provided by engagement with the analyte upon a certain body, or volume of the detection medium beyond its surface. In this way, the body or volume of the detection medium may absorb, adsorb, dissolve, intercalate or otherwise take up an analyte within its body. In this way, body or volume of the detection medium may have a capacity to absorb, adsorb, dissolve, intercalate or otherwise collect an analyte within its body.

The second response phase thus may provide a second change to the dielectric or electrical resistance characteristics of the sensor device formed within the groove. The second change of the dielectric or electrical resistance characteristics may be suitably detected by measuring, for example, one or more of the capacitance, resistance or impedance across the device, as is set out in more detail herein. In the second response phase, a second rate of change is measured.

In this way, the detection medium of electronic sensor device of the invention may be arranged to provide both a surface engagement and a bulk engagement with the analyte. That is, by providing the detection medium within a groove of a substrate, it may be possible to provide a sensor including a detection medium which contactingly engages an analyte and provides a signal response including first and second response phases.

Advantageously, by providing a detection medium within a groove, there may be provided a sensor adapted to provide a first response phase as an analyte contactingly engages with a surface.

Furthermore, there may be also provided a sensor device adapted to provide a subsequent, second response phase as the analyte is absorbed, adsorbed, dissolved, intercalated or otherwise taken up within the detection medium. Preferably, the second response phase may be provided by an analyte reaching an equilibrium absorption, adsorption, dissolution or uptake with the detection medium. In this way, the second phase response may correspond to a second rate of change tending towards zero. In other words, the second response phase may be provided as a change of electrical characteristic until an equilibrium point is reached at which there is a substantially constant amount of analyte within the detection medium.

Aptly, said detection medium may include a medium surface and a medium body, and wherein said first response phase is provided in response to an engagement of the analyte with said medium surface.

In this way, the surface effect of the analyte upon the detection medium may be provided by engagement of the analyte with the medium surface of the detection medium.

Aptly, said second response phase may be provided in response to an engagement of the analyte within said medium body.

In this way, the bulk effect of the analyte upon the detection medium may be provided by engagement of the analyte with the medium body of the detection medium.

Due to the signal response provided as a first and second phase response, a surprising advantage has been noted by the inventors. Thus, while the sensitivity of a sensor device, that is the speed with which an analyte engages a sensor to provide a response signal, may be improved by providing a groove with large surface area, such a device is substantially only capable of providing an unstable first phase response. In other words, such a device may provide unreliable electrical characteristic measurements, because an analyte which engages its surface may do so in only a transient manner.

In contrast, by providing a sensor device of an aspect of the invention, in which grooves include a groove depth, a sensor device may provide a second phase response. Stated differently, by providing a sensor device with a medium body, a sensor device may provide a second phase response. In this way, the signal response of a sensor is made more reliable. Increased reliability of a sensor may be achieved by providing grooves with a suitable aspect ratio (that is the ratio of groove width to groove depth). Without being bound to theory, the inventors believe the reliability may be improved because the initial surface engagement of an analyte with the detection medium, thereby providing a first phase response, is followed by a subsequent bulk engagement of the analyte with the detection medium, thereby providing a second phase response.

Empirical data showing the first and second phase responses are described herein within reference to FIGS. 6 to 12.

Aptly, said medium body may include a portion of the substrate forming the profile of the at least one groove.

In this way, the electronic sensor device may simply be provided using a surrounding fluid as a medium. That is, the device may be provided with a groove including respective first and second electrically non-insulating portions on the first and second faces, and further including air within the groove. In this way, in an initial phase, in the absence of an analyte, air within the groove may provide a dielectric or an resistive element within the groove and an electrical characteristic, such as capacitance or resistance may be measured across the sensor. With this example arrangement, contact with the sensor by an analyte causes replacement of a portion of the air within the groove by an analyte. The first phase response may thus be provided by contacting engagement of the analyte with the first and second electrically non-insulating portions of the groove.

A second phase response may also be provided by this example arrangement. The second phase response may be provided by adsorption of the analyte into the substrate which forms at least a portion of the cross-sectional profile of the groove.

That is, the second phase response may be provided by adsorption of the analyte into at least the substrate exposed within the groove in the space providing the electrical separation between first and second electrically non-insulating portions.

Aptly, said profile of said at least one groove may have a first characteristic shape and, in use, in second response phase said profile may deform to a second characteristic shape. In this way, engagement of an analyte with the sensor may provide adsorption of the analyte by the substrate within the groove and, thereby, a change to the groove cross-sectional shape. A change to the groove profile, or cross-sectional shape may provide a second phase response. Stated differently, deformation of the profile may change an electrical characteristic of the sensor device to provide a second phase response. Without being bound to theory, the inventors believe the deformation may be due at least in part to absorption, or adsorption of the analyte by the substrate exposed within the groove.

Aptly, said signal response may include a change in at least one predetermined electrical characteristic of said at least one groove.

Aptly, said predetermined electrical characteristic may be an impedance, a resistance or a capacitance of said at least one groove.

In these ways, the first and second phase responses may be readily measured by suitable detectors within an electrical circuit, such as described herein.

Aptly, within said first response phase, said predetermined electrical characteristic may change at a first rate and, within said second response phase, said predetermined electrical characteristic may change at a second rate.

Aptly, said first rate change may be of a greater magnitude than said second rate.

In these ways, it is possible to measure and distinguish a first phase response from a second phase response. Furthermore, said first and second phase responses provide a reliable characteristic signal response due to engagement of the sensor with an analyte or series or analytes.

Aptly, said first response phase may last for a first time interval and said second response phase lasts for a second time interval. More aptly, said first time interval may be less than 1 second and said second time interval may be less than 60 seconds.

Alternatively, said first time interval may be less than 60 seconds and said second time interval may be less than 600 seconds. Preferably, said first time internal may be less than 40 seconds and said second time interval may be less than 180 seconds. Yet more preferably, said first time internal may be in a range of from 20 to 40 seconds and said second time interval may be in a range of from 80 to 120 seconds.

In these ways, a detection medium may be tuned to provide characteristically distinguishable first and second phase responses. That is, the sensor may be adapted, for example, by selecting an appropriate groove aspect ratio, in order to provide a first phase response which is clearly and distinctively differentiated from the second phase response.

The first or second phase response may also be clearly and distinctively differentiated from other signal responses, for example measurement of an electrical characteristic prior in the absence of an analyte.

Furthermore, depending on the nature of the analyte, the sensor may be adapted to provide an increased rate of change in the first phase response.

Additionally, or alternatively, the sensor may be adapted to provide a second phase response in which the measured electrical characteristic of the sensor is characteristically changed from the measured electrical characteristic in the absence of an analyte.

Aptly, said groove width may be less than or equal to 100 μm, more aptly, is less than or equal to 40 μm, and yet more aptly is within the range 0.5 μm to 10 μm.

Aptly, a groove aspect ratio may be defined as the ratio of said groove depth to said groove width, such that said groove aspect ratio may be within the range 0.1:1 to 50:1. More aptly the groove aspect ratio may be within the range 1:1 to 6:1.

Aptly, said profile may be any one of: U-shaped, V-shaped, asymmetrically V-shaped, rounded, semi-circular, or square shaped.

Aptly, said profile may include a flat base.

Aptly, said first or second electrically non-insulating portion may extend from said respective first or second face onto said surface of said substrate.

Aptly, said first or second electrically non-insulating portion may include a coating. In certain examples, the first electrically non-insulating portion is substantially the same composition as second electrically non-insulating portion. In certain examples, the first electrically non-insulating portion may differ to second electrically non-insulating portion.

An electrically non-insulating portion, that is one or both of the respective first and second electrically non-insulating portions, may be provided from any suitable composition, including electrical conductors or semiconductors. More aptly, an electrically non-insulating portion may be provided as a carbon, a metal or a non-metal, such as a metal oxide coating. A suitable metal for an electrically non-insulating portion may be provided as a coating formed from any one of aluminium, gold, silver, nickel, titanium, manganese, or any other appropriate metal. A suitable non-metal may be any metal oxide provided as a coating formed thereon as will be known to the skilled person. Non-limiting examples of metal oxides include an aluminium oxide, a titanium oxide, an iron oxide or a manganese oxide.

Aptly, the first face and/or the second face of the respective groove or grooves may be coated with a first material and/or a second material by an off-axis directional coating process. This provides the advantage that certain faces of the groove or grooves can be selectively coated during manufacture.

Alternatively, or additionally, the first or second electrically non-insulating portion may include or may be a discontinuous electrically non-insulating coating of the substrate. That is, an electrically non-insulating coating may be deposited on the substrate to provide one or more electrically non-insulating portions.

The electrically non-insulating portion may be formed by etching, or removing, a portion of another coating of the substrate to expose an electrically non-insulating coating. In some embodiments, the electrically non-insulating portion may be formed by masking of a region of a connecting portion on the surface of the substrate during manufacture. Thus, a region of the connecting portion may be devoid of electrically conductive material.

Aptly, said at least one groove may include a series of grooves. That is, in certain embodiments the electronic sensor may include a first series of grooves.

The first series of grooves may include any number of grooves, i.e. it may include a first terminal groove and a second terminal groove.

Each groove of the first series of grooves may extend across the transverse direction of the substrate.

Each groove of the first series of grooves may extend in parallel.

Any number of grooves may be provided within the first series of grooves between a first terminal groove and a second terminal groove. The first terminal groove may terminate, or form a terminus of, the first series of grooves at one end, or a first end, for example a distal end. The second terminal groove may terminate, or form a terminus of, the first series of grooves at another end, or a second end, for example a proximal end.

The distal end and the proximal end denote ends of the first series of grooves across the web direction of the substrate.

The sensor device may further include a second series of grooves. The second series of grooves may include any number of grooves, i.e. it may include a first terminal groove and a second terminal groove.

Each groove of the second series of grooves may extend across the transverse direction of the substrate.

Each groove of the second series of grooves may extend in parallel.

Each groove of the second series of grooves may be in parallel with each groove of the first series of grooves.

Any number of grooves may be provided within the second series of grooves between a first terminal groove and a second terminal groove. The first terminal groove may terminate, or form a terminus of, the second series of grooves at one end, or a first end, for example a distal end. The second terminal groove may terminate, or form a terminus of, the second series of grooves at another end, or a second end, for example a proximal end.

The distal end and the proximal end denote ends of the second series of grooves across the web direction of the substrate.

The first terminal groove and the second terminal groove may each be proximal to a connecting portion on a surface of the substrate. Each connecting portion provides a means of electrical connecting a groove, for example a terminal groove. In certain examples a connecting portion provide a groove with an electrical connection within an electrical circuit. In this way, a respective first and second connecting portions may provide a first and a second electrode of the sensor device.

Additionally, or alternatively, the second terminal groove of the first series of grooves and the first terminal of the second series of grooves may be separated by, or spaced apart by, the connecting portion. That is, there may exist an electrical connection, provided by a connecting portion, between the first series of grooves and the second series of grooves.

Aptly, the at least one groove may comprise a single groove having parallel sections adjoined at opposing edges, thereby forming a substantially repeating S-shape.

Aptly, each groove of said series of grooves may be from 5 mm to 1000 mm long.

Aptly, a combined length of said series of grooves may be greater than 100 m in length, more aptly greater than 1,000 m in length. Yet more aptly, a combined length of said series of grooves may be greater than 10,000 m in length and even more aptly between 10,000 m and 60,000 m in length.

Aptly, a second face of a first groove of the said series of grooves may be in electrical communication with a first face of a second groove of said series of grooves.

Aptly, said series of grooves may be aligned on said substrate surface with a predetermined groove density. As used herein, groove density may be defined as the ratio of total groove surface area to total substrate area. Aptly, said groove density may be within the range 50-80%, more preferably within the range 60-70%.

The substrate may comprise a curable resin and in particular a UV curable resin. Aptly, The substrate may comprise one or more of an acrylic resin coated onto polyvinyl chloride (PVC), acrylic resin coated onto polyethylene terephthalate (PET), acrylic resin coated onto polyethylene naphthalate (PEN), a biopolymer coated onto polyvinyl chloride (PVC), a biopolymer coated onto polyethylene terephthalate (PET) and a biopolymer coated onto polyethylene naphthalate (PEN). More aptly a substrate may be impregnated or doped with an electrically non-insulating material. In certain examples, a substrate may be impregnated or doped to at least 30% by volume, more aptly to at least 37% by volume.

Aptly, said detection medium is conformally deposited onto at least a portion of said profile. In this way, the detection medium may protectively cover a first or second face, or a first or second electrically non-insulating portion

Aptly, said detection medium may partially fill, substantially fill, or overfill said at least one groove.

Aptly, said detection medium may include a binder and an active component in a mixed state.

Aptly, said binder and said active component may be uniformly dispersed.

Aptly, the signal response may be provided in response to an engagement of the analyte with said active component.

Aptly, said active component may be provided as a particulate or as a liquid.

Aptly, said binder may be adapted to bind said active component within said detection medium.

Aptly, said profile or said detection medium may be further adapted such that, in use, in response to a discontinued engagement of the analyte with the detection medium, a recovery signal may be provided. More aptly, the recovery signal may include a first recovery phase and a subsequent second recovery phase.

Aptly, said first response phase may be provided in response to discontinued engagement of the analyte from a surface of said detection medium.

Aptly, said second response phase may be provided in response to dissociation of the analyte from said medium body.

In these ways, the electronic sensor may be readily adapted to provide a response signal due the absence or reduction of the analyte with the sensor.

According to a further aspect of the invention, there is provided an analyte detection apparatus comprising:
an electronic sensor including a substrate including at least one groove, said groove including a first face and a second face, said groove having a cross-sectional profile including at least a groove depth within the substrate and a groove width at a surface of said substrate;
said first face including a first electrically non-insulating portion and said second face including a second electrically non-insulating portion wherein, within said profile, said first electrically non-insulating portion is electrically separated from said second electrically non-insulating portion;
a detection medium provided within said groove, arranged to contactingly engage said first and second electrically non-insulating portions, and adapted to be contactingly engaged by an analyte, and
wherein said profile or said detection medium is adapted such that, in use, a signal response is provided comprising a first response phase and a subsequent second response phase in response to an engagement of the analyte with the detection medium, a detector,
wherein said first electrically non-insulating portion of said at least one groove forms a first electrode and said second electrically non-insulating portion of said at least one groove forms a second electrode, and
wherein said first and second electrodes are electrically connected to said detector such that said detector detects and measures said signal response.

Aptly, said detector detects and measures said recovery signal.

Aptly, the electronic sensor device may be referred to as a two terminal device. The first and second series of grooves may be referred to as cascaded groove structures. In use the device may be fabricated in a series arrangement and operated in a parallel or a combined series and parallel arrangement.

According to a yet further aspect of the invention, there is provided a method of producing an electronic sensor device, comprising the steps of:
providing said substrate;
forming said at least one groove within said surface thereby providing a first face, a second face and a profile including at least a groove depth within the substrate and a groove width at a surface of said substrate, wherein said first face includes a first electrically non-insulating portion and said second face includes a second electrically non-insulating portion wherein, within said profile, said first electrically non-insulating portion is electrically separated from said second electrically non-insulating portion, and
providing a detection medium within at least a portion of said profile so as to contactingly engage said first and second electrically non-insulating portions, wherein said profile or said detection medium is adapted such that, in use, a signal response is provided comprising a first response phase and a subsequent second response phase in response to an engagement of the analyte with the detection medium.

Aptly, said method further includes providing said detection medium by mixing an active component (e.g. a filler) with a binder.

During the manufacture of electronic sensor devices, an off-axis directional coating process may be used, in which at least one face of the grooves or series of grooves is selectively coated. This is particularly useful for roll-to-roll manufacture of such devices, as the manufacturing process can be carried out as a continuous process, rather than batch process. In such cases, the opposing face of the grooves casts a shadow onto the face to be coated, such that only a portion of the face to be coated can be coated by the incoming material. This is known as the “shadowing effect”. Thus, the shadowing effect governs the amount of material deposited on a face of the grooves. The shadowing effect can be modified by increasing or decreasing the angle of the off-axis directional coating.

### BRIEF DESCRIPTION OF THE DRAWINGS

Embodiments of the invention are now described, by way of example only, hereinafter with reference to the accompanying drawings, in which:

FIG. 1. shows a cross-sectional view of a first example embodiment electronic sensor device according to an aspect of the invention;

FIG. 2. shows a cross-sectional view of a second example embodiment electronic sensor device according to an aspect of the invention;

FIG. 3. shows a cross-sectional view of a third example embodiment electronic sensor device according to an aspect of the invention;

FIG. 4. shows a perspective view of an example embodiment electronic sensor according to an aspect of the invention provided as a two terminal device for electrical connection to a detector;

FIG. 5. shows (a) a model signal response under test conditions and (b) the metric definitions of the model signal response, of an example embodiment device according to an aspect of the invention;

FIG. 6. shows empirical data of a capacitance signal response in response to liquid water measured by the two terminal device of FIG. 4 incorporating (a) both a first detection medium, (b) a first detection medium alone and (c) a second detection medium alone;

FIG. 7. shows empirical data of a capacitance signal response of a further example embodiment electronic sensor device under test conditions in response to an atmosphere including (a) 5% carbon dioxide gas and (b) initially 2% and subsequently 5% carbon dioxide gas;

FIG. 8. shows empirical data of an impedance signal response of the device of FIG. 9 in response to an atmosphere of 5% carbon dioxide gas;

FIG. 9. shows an example method of forming an electronic sensor device according to an aspect of the invention;

FIG. 10. shows empirical data of a capacitance signal response in response to liquid water measured by the two terminal device of FIG. 4 incorporating a third detection medium;

FIG. 11. shows empirical data of a resistance signal response in response to 5% hydrogen gas measured by the two terminal device of FIG. 4 incorporating a fourth detection medium; and

FIG. 12. shows empirical data of a resistance signal response in response to 0.8-0.9% hydrogen gas measured by the two terminal device of FIG. 4 incorporating a fifth detection medium; and.

In the drawings, like reference numerals refer to like parts.

### DETAILED DESCRIPTION

Certain terminology is used in the following description for convenience only and is not limiting. The word ‘upper’ designates a direction in the drawings to which reference is made and are with respect to the described component when assembled and mounted.

Further, as used herein, the terms ‘connected’, ‘attached’, and ‘mounted’ are intended to include direct connections between two members without any other members interposed therebetween, as well as, indirect connections between members in which one or more other members are interposed therebetween. The terminology includes the words specifically mentioned above, derivatives thereof, and words of similar import.

Further, unless otherwise specified, the use of ordinal adjectives, such as, ‘first’, ‘second’, ‘third’ etc. merely indicate that different instances of like objects are being referred to and are not intended to imply that the objects so described must be in a given sequence, either temporally, spatially, in ranking or in any other manner.

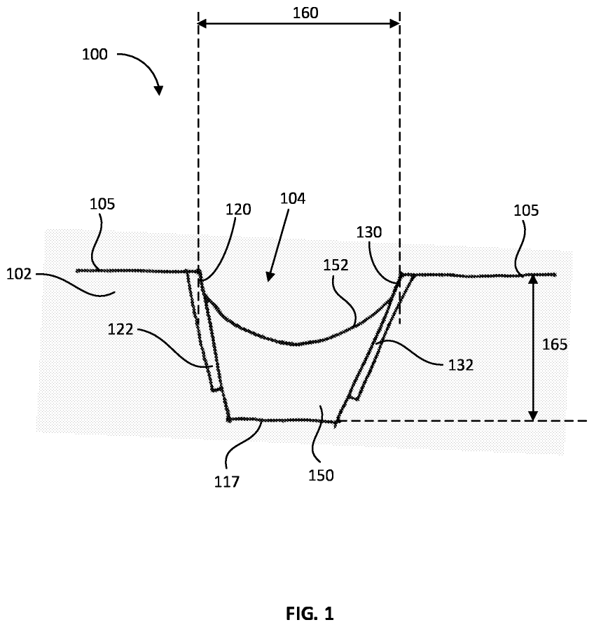

Referring now to FIGS. 1(a) and 1(b), there is shown a cross-sectional view of an electronic sensor device 100 according a first aspect of the invention. The example of FIG. 1(a) and (b), the sensor device 100 includes a substrate 102 having a groove 104, which includes a first face 120 and a second face 130, and which has a cross-sectional profile including at least a groove depth 165 within the substrate and a groove width 160 at a surface 105 of the substrate 102.

The first face 120 includes a first electrically non-insulating portion 122, and the second face 130 includes a second electrically non-insulating portion 132 wherein, within the profile, the first electrically non-insulating portion 122 is electrically separated from the second electrically non-insulating portion 132.

A detection medium 150 is provided within said groove, arranged to contactingly engage said first and second electrically non-insulating portions 122, 132, and adapted to be contactingly engaged by an analyte.

Either the profile of the groove 104 or the detection medium 150 is adapted such that, in use, a signal response is provided including a first response phase and a subsequent second response phase in response to an engagement of the analyte with the detection medium 150.

The groove 104 is formed such that it extends across the surface 105 of the substrate 102 from a proximal end to distal end. The groove 104 extends linearly across the substrate 102. In certain other examples, the groove may extend across the substrate in any suitable orientation or configuration, such as a Z-shape, a zig zag or a curved arrangement.

The groove 104 is formed with a cross-sectional profile. In the example shown, the cross-sectional profile includes first and second faces 122, 132, opposingly arranged and extending into the substrate 102. The first and second faces 122, 132 are spaced apart by a base 117.

The profile forms a generally U-shaped groove including a groove width 160 determined by the distance between the first and second faces 122, 132 at the surface 105 of the substrate. The profile includes at the depth 165 determined by the vertical distance that the groove 104 extends within the substrate 102.

A profile further includes a groove aspect ratio. The groove aspect ratio as used herein is defined as the ratio between the width of the groove at the substrate surface to the depth of the groove within the substrate. In the example shown in FIG. 1, the groove 104 is provided with a groove width 160 of 5 μm, and a groove depth of 10 μm. The groove aspect ratio is 2:1.

The first and second faces 122, 132 of the groove 104 are substantially of equal length and extend from the surface 105 at a common angle to a reference axis extending orthogonal to the surface 105 of the substrate 102.

In certain other examples the groove profile may be any suitable shape including, but not limited to V-shaped, rounded, semi-circular, or square shaped. The groove profile may be symmetrical or asymmetrical about the reference axis. That is, for example, a profile may be a V-shaped with faces of different lengths, or with different angles to the reference axis.

The first face 120 is provided with a first electrically non-insulating portion 122. The first electrically non-insulating portion 122 extends along the length of the first face 120 within the groove 104. In the example shown, the first electrically non-insulating portion 122 is a metallised aluminium coating.

The first electrically non-insulating portion 122 extends a portion of the depth of the first face 120, from the surface 105 to a distance above the groove base 117.

The second face 130 is provided with a second electrically non-insulating portion 132. The second electrically non-insulating portion 132 extends along the length of the second face 130 within the groove 104. In the example shown, the second electrically non-insulating portion 132 is a metallised aluminium coating.

The second electrically non-insulating portion 132 extends a portion of the depth of the first face 120, from the surface 105 to a distance above the groove base 117.

The first and second electrically non-insulating portions 122, 132 are electrically separated across the groove 104. That is, first and second electrically non-insulating portions 122, 132 are spaced apart across the base 117 of the groove 104.

In the example shown in the FIG. 1, the first electrically non-insulating portion 122 is substantially the same composition as second electrically non-insulating portion 132. In certain examples, the first electrically non-insulating portion 122 may differ to second electrically non-insulating portion 132.

The first and second electrically non-insulating portions 122, 132 may be provided from any suitable composition, including electrical conductors or semiconductors. Preferably, electrically non-insulating portion 122 and second electrically non-insulating portion 132 may be provided as a carbon, or a metal or a metal oxide coating. Even more preferably, electrically non-insulating portion 122 and second electrically non-insulating portion 132 may be provided as coatings of aluminium, gold, silver, nickel, titanium, manganese or oxides thereof, such as an aluminium oxide, a titanium oxide or a manganese oxide.

The first and second electrically non-insulating portions 122, 132 may be provided as coatings or depositions onto the respective groove faces. A respective coating may be applied using any suitable techniques, such as an off-axis directional coating process. A respective coating may be applied using a vapour deposition technique. Alternatively, the substrate may be formed of an electrically non-insulating material such that the first and second electrically non-insulating portions may be provided by forming of the respective groove faces within the substrate.

A detection medium 150 is provided within the groove 104. The detection medium 150 is deposited within the groove such that the detection medium 150 contactingly engages the first electrically non-insulating portion 122 and contactingly engages the second electrically non-insulating portion 132.

The detection medium 150 partially fills the groove 104. That is, the detection medium 150 covers the base 117 of the groove 104 such that it extends partially up the first and second face 120, 130, thereby partially covering the first and second electrically non-insulating portions 122, 132. The detection medium 150 is thereby provided with a surface 152 contained within the groove 104. That is, the surface 152 of the detection medium 150 is exposed within the groove, such that an agent, for example an analyte, entering the groove 104 engages with the detection medium 150.

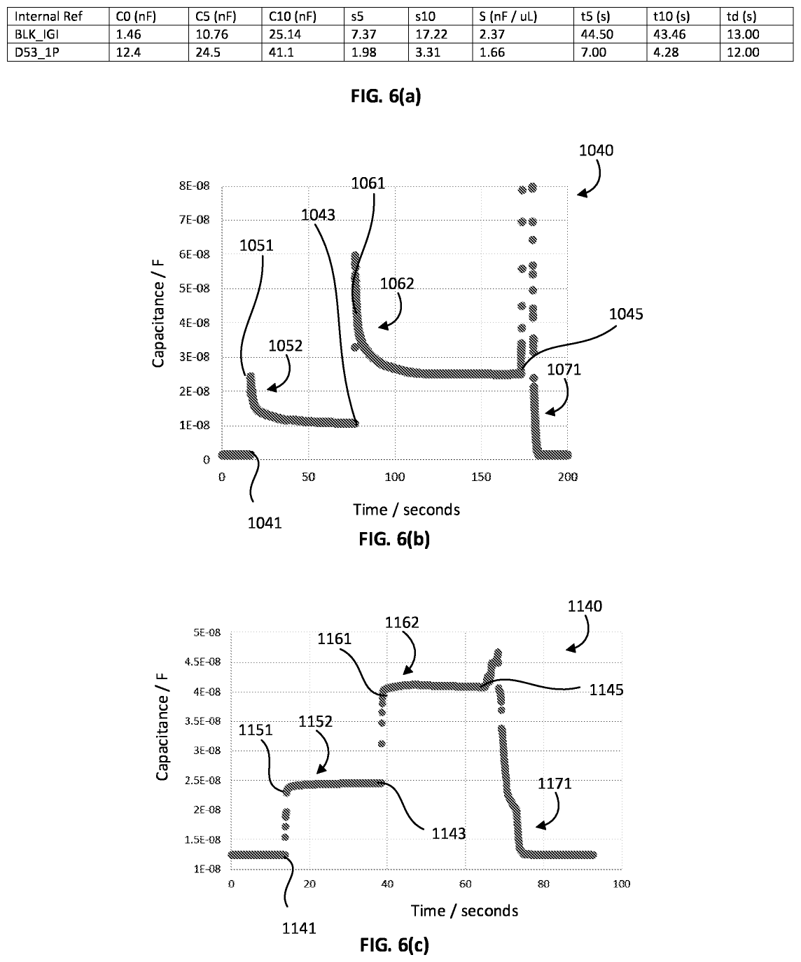

In use, the electronic sensor device 100 is connected within an electrical circuit, such as a two terminal device including a detector, as described herein. By applying an electrical potential difference across the sensor device 100, an electrical characteristic of the sensor device can be detected and measured using a suitable detector, as explained herein. In certain examples, the electrical characteristic is the capacitance measured across the groove 104. Optionally, the electrical characteristic is the impedance or resistance measured across the groove 104. Further details of a measurement of an electrical characteristic is provided herein in relation to FIGS. 6 to 10.

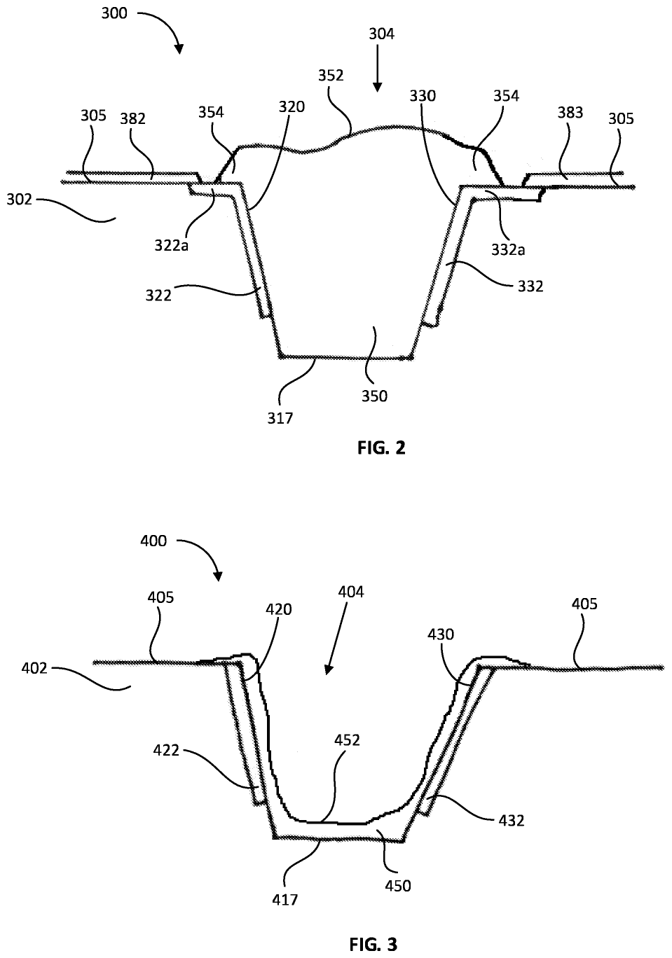

Referring now to FIG. 2, there is shown a second example embodiment of an electronic sensor device 300. Where the features are the same as the previous example, the reference numbers are also kept the same, but with a “3” as the initial digit.

Further, FIG. 2 shows a cross-sectional view of sensor 300 including a groove 304 within a substrate 302 and including first and second faces 320, 330. The profile of the groove 304 is substantially the same as the profile of the groove 104 of the example embodiment shown in FIG. 1, so, for brevity, the details are not repeated here.

The first face 320 of the groove 304 is provided with a first electrically non-insulating portion 322. The first electrically non-insulating portion 322 extends the length of the first face 320 within the groove 304. In the example shown, the first electrically non-insulating portion 322 is a metallised aluminium coating.

The first electrically non-insulating portion 322 extends a portion of the depth of the first face 320, from the surface 305 to a distance above the groove base 317. The first electrically non-insulating portion 322 further extends a distance along the surface 305 10 of the substrate to provide a first contacting portion 322a.

A surface 305 of the substrate 302 is provided with a first coating 382. The first coating 382 extends away from an upper edge of the first face 320 of the groove. That is, the first coating 382 is provided in contacting engagement with the contacting portion 322a of the first electrically non-insulating portion 322. The first electrically non-insulating portion 322 is thus able to form a first electrode. In this way, the first electrically non-insulating portion 322 may be electrically connected to a first terminal of a two terminal device, as explained further herein.

The second face 330 of the groove 304 is provided with a second electrically non-insulating portion 332. The second electrically non-insulating portion 332 extends the length of the second face 330 within the groove 304. In the example shown, the second electrically non-insulating portion 332 is a metallised aluminium coating.

The second electrically non-insulating portion 332 extends a portion of the depth of the first face 320, from the surface 305 to a distance above the groove base 317. The second electrically non-insulating portion 332 further extends a distance along the surface 305 of the substrate 302 to provide a second contacting portion 332a.

A surface 305 of the substrate 302 is provided with a second coating 383. The second coating 383 extends away from an upper edge of the second face 330 of the groove. That is, the second coating 383 is provided in contacting engagement with the contacting portion 332a of the second electrically non-insulating portion 332. The second electrically non-insulating portion 332 is thus able to form a second electrode. In this way, the second electrically non-insulating portion 332 may be electrically connected to a second terminal of a two terminal device, as explained further herein.

A detection medium 350 is provided within the groove 304. The detection medium 350 is deposited within the groove such that the detection medium 350 contactingly engages the first electrically non-insulating portion 322 and contactingly engages second electrically non-insulating portion 332.

The detection medium 350 overfills the groove 304. That is, the detection medium 350 fills the cavity within the groove such that the groove surface 352 extends above the plane of the surface 305 of the substrate 302. That is, the surface 352 of the detection medium 350 is exposed above the groove, such that an agent, for example an analyte, approaching the groove 304 engages with the detection medium 350.

In use, the electronic sensor device 300 is connected within an electrical circuit, such as a two terminal device including a detector, using the first and second electrodes. An electrical characteristic of the sensor device 300 can be detected and measured using a suitable detector in same manner as the sensor device of the example embodiment of FIG. 1.

Referring now to FIG. 3, there is shown a third example embodiment of an electronic sensor device 400. Where the features are the same as the previous examples, the reference numbers are also kept the same, but with a “4” as the initial digit.

Further, FIG. 3 shows a cross-sectional view of sensor 400 including a groove 404 within a substrate 402 and including first and second faces 420, 430. The groove profile as well as the first and second electrically non-insulating portions 422, 432 are substantially the same as the corresponding features of the groove 104 of the example embodiment shown in FIG. 1, so, for brevity, the details are not repeated here.

A detection medium 450 is provide within the groove 404 so that it conformally covers the groove profile. That is, the detection medium 450 covers the first face 420, the second face 430 and the base 417 of the groove 404 with a layer of detection medium 450 material so that the groove is not filled with detection medium 450.

At the respective upper edges of the first and second faces 420, 430, the detection medium 450 extends out of the groove 404 thereby covering the boundary of the first and second electrically non-insulating portions 422, 432 with the surface 405. In this way, the detection medium 450 is arranged to provide a covering to the first and second electrically non-insulating portions 422, 432 so that they are not exposed to the analyte. The detection medium 450 is arranged to encapsulate the first and second electrically non-insulating portions 422, 432, thereby providing a protective layer.

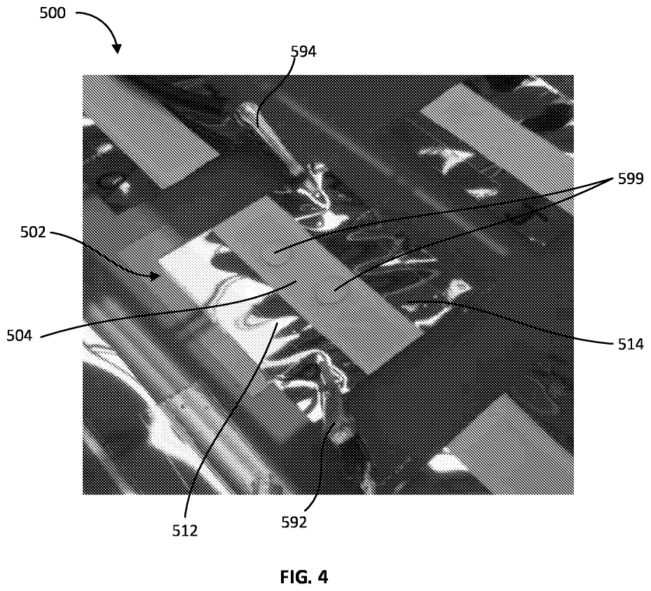

Referring now to FIG. 4, there is shown a perspective view of an example embodiment electronic sensor 500 provided as a two terminal device for electrical connection to a detector. Where the features are the same as the previous examples, the reference numbers are also kept the same, but with a “5” as the initial digit.

The electronic sensor 500 includes a substrate 502 formed with region including a series of grooves 504.

The substrate 502 includes a first area provided spacers and channels arranged with the series of groove 504 as described herein. The first area is formed as a first terminal 512 extending between the series of grooves 504 and a first edge of the substrate 502. The first terminal 512 is electrically connected to a first connector 592 mounted thereto.

The substrate 502 includes a second area provided spacers and channels arranged with the series of groove 504 as described herein. The second area is formed as a second terminal 514 extending between the series of grooves 504 and a second, opposing edge of the substrate 502. The second terminal 514 is electrically connected to a first connector 594 mounted thereto.

The substrate 502 is shown with two portions of an analyte 599 deposited thereon. In the example shown, the analyte 599 is provided as two liquid droplets contactingly engaging the series of grooves 504.

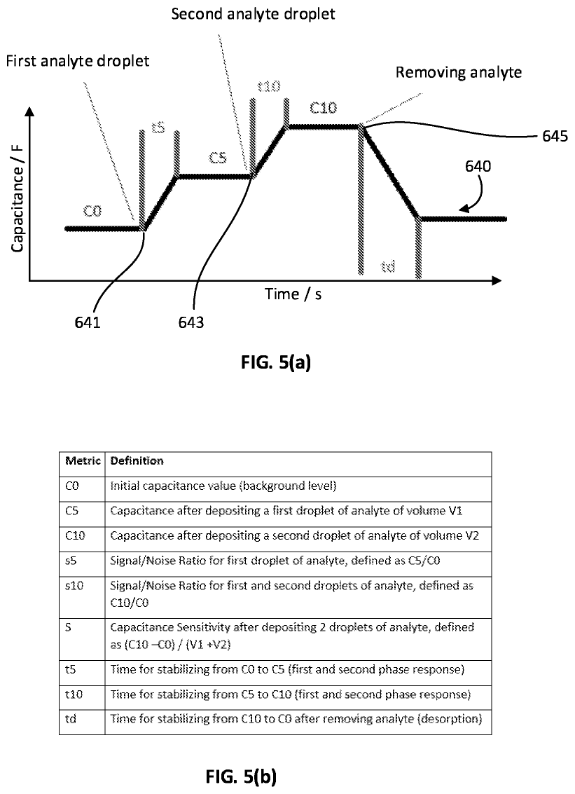

The first and second terminals 512, 514 are electrically connected to a detector (not shown) via the first and second connectors 592, 594. That is the first and second terminals 512, 514 form an electrical circuit with a detector. The detector is suitable chosen to detect and measure an electrical characteristic of the series of grooves 504, as is described in more detail in reference to FIG. 5. In this way, the sensor device 500 is formed as a two terminal device electrically connected to a detector.

Referring now to FIG. 5, there is shown a model signal response using, for example, the sensor 500 and electrical detector as shown in FIG. 4. Particularly, there is shown in FIG. 5(a) a model capacitance signal response 640 measured over time as a first analyte portion and then a second analyte portion contactingly engage with an electronic sensor device. In the model signal response, the analyte portions are droplets of liquid water, although the signal response may be indicative of a range of analytes in fluid form.

The metric definitions taken from the model signal response 640 are shown in FIG. 5(b). An initial capacitance value (C0) corresponds to a background signal response. That is, C0 corresponds to a capacitance value measured prior to engagement of an analyte with the sensor device. In this way, C0 represents a constant capacitance signal response of the sensor before an analyte is present.

A first analyte droplet engages 641 the sensor. Engagement of the first analyte droplet provides a first signal response in the sensor. The first analyte has a volume V1. After the first signal response the first droplet remains engaged with the sensor. The first signal response includes first and second phase responses corresponding to a surface effect of the first analyte droplet on the sensor, and a bulk effect of the first analyte droplet as described herein.

The first phase response of the first signal response has a duration of time interval t5, after which the detector measures a first characteristic capacitance C5. C5 is a different capacitance to the baseline capacitance C0. C5 represents a second phase response of the first signal response. That is C5 represents a static capacitance signal response following engagement of the first analyte droplet with the sensor.

A second analyte droplet engages 643 the sensor. Engagement of the second analyte droplet provides a second signal response in the sensor. The second analyte has a volume V2. After the second signal response the second droplet remains engaged with the sensor. The second signal response includes first and second phase responses corresponding to a surface effect of the second analyte droplet on the sensor, and a bulk effect of the second analyte droplet as described herein.

The first phase response of the second signal response has a duration of time interval t10, after which the detector measures a second characteristic capacitance C10. C10 is a different capacitance to both the baseline capacitance C0 and the first characteristic capacitance C5. C10 represents a second phase response of the second response signal. That is, C10 represents a static capacitance signal response following engagement of the second analyte droplet engages the sensor.

Additionally, or alternatively, the second phase response of either the first or second signal responses may include a change in the measured electrical characteristic. That is, a second phase response is not limited to only a static electrical characteristic, such as capacitance. In certain examples, a second phase response may include a change of electrical characteristic over time caused by the bulk effect of an analyte upon a sensor. In this way, within the second phase response, the rate of change may decrease to zero over a predetermined time. In other words, within a second phase response the electrical characteristic may tend towards a static measurement as the analyte approaches an equilibrium with the bulk volume of the detection medium.

The model response 640 also includes removal 645 of the first and second analyte droplets from the sensor. Removal of the analyte droplets provides a recovery signal response in the sensor. The recovery signal response includes first and second phase responses corresponding to a discontinuation of the surface effect by the analyte on the sensor, followed by a discontinuation of the bulk effect of the analyte on the sensor.

The recovery signal response has a duration of time interval td, after which the detector measures the baseline capacitance C0.

A signal to noise ratio, s5, for a signal response provided by a first analyte droplet is defined herein as C5/C0. A signal to noise ratio, s10, fora signal response provided by a first and second analyte droplets is defined herein as C10/C0. A capacitance sensitivity S to first and second analyte droplets is thus defined herein as:

$$S = \frac{( C 10 - C 0 )}{( V 1 + V 2 )}$$

The first signal response of model response shown in FIG. 5, including the respective first and second phase responses may provide be sufficient to indicate that the sensor has detected an analyte. That is, the first signal response on its own may provide a reliable, measurable change in electrical characteristic, in this case capacitance, to indicate an analyte has been detected. The second signal response may not be required in order to indicate that the sensor has detected an analyte. Thus, the second signal response shown in FIG. 5 is provided to illustrate that a sensor as described herein may provide multiple signal responses due to engagement with increasing volumes of analyte.

Referring now to FIG. 6, there is shown empirical data of a capacitance signal response measured by the two terminal device shown in FIG. 6 when incorporating two different sensor devices according to an aspect of the invention.

The two sensors are identified as BLK_IGI and D53_P. The sensors are each formed from a reference substrate including a series of grooves having a groove width of 5.3 μm and a groove depth of 10 μm. The reference substrate has a groove density of 66%. The first and second faces of the grooves are formed with metallised faces. That is the grooves of the reference substrate include the first and second electrically non-insulating portions formed as layers of aluminium approximately 100 nm thick.

The sensor BLK_IGI includes air as a detection medium. FIG. 6(b) shows the signal response 1040 of sensor BLK_IGI in response to a first and second droplet of analyte, in this case liquid water, as explained in reference to FIG. 4. The metrics of signal response are provided in the table shown in FIG. 6(a), corresponding the metrics of the model response explained with reference to FIG. 5.

A first droplet engages 1041 with the sensor providing a first signal response. The first signal response includes a first phase response 1051 in which the capacitance increases almost instantaneously from a baseline of around 0 F to around 2.5×10⁻⁸ F, a change of around 2.5×10⁻⁸ F or 25 nF. The signal to noise ratio of the first phase response, s5, was measured as 7.37. The first phase response corresponds to a surface effect of the first droplet on the sensor.

A second phase response 1052 is provided in which the capacitance decreases to a first characteristic capacitance C5. The second phase response corresponds to a bulk effect of the first droplet on the sensor. The time for the first signal response, t5, is 44.5 seconds.

A second droplet engages 1043 with the sensor providing a second signal response. The second signal response includes a first phase response 1061 in which the capacitance increases almost instantaneously from around 1.0×10⁻⁸ F to around 6.0×10⁻⁸ F, a change of around 5.0×10⁻⁸ F or 50 nF. The signal to noise ration of the first phase response, s10, was measured as 17.22. The first phase response corresponds to a surface effect of the second droplet on the sensor.

A second phase response 1062 is provided in which the capacitance decreases to a second characteristic capacitance C10. The second signal response corresponds to a bulk effect of the first droplet on the sensor. The time for the second signal response, t10, is 43.46 seconds.

The first and second droplets are removed 1045 from the sensor, providing a recovery signal response 1071.

The sensor D53_1P includes a detection medium including Dupont Luxprint 8153 the further details of which are provided herein. The detection medium is provided as a single coat of the grooved area of the reference substrate.

FIG. 6(c) shows the signal response 1140 of sensor D53_1P measured in response to a first and second droplet of analyte, in this case liquid water, as explained in reference to FIG. 4. The metrics of signal response are provided in the table shown in FIG. 6(a), corresponding the metrics of the model response explained with reference to FIG. 5.

A first droplet engages 1141 with the sensor providing a first signal response. The first signal response includes a first phase response 1151 in which the capacitance increases almost instantaneously from around 0 F to around 2.5×10⁻⁸ F. The signal to noise ration of the first phase response, s5, was measured as 1.98. The first phase response corresponds to a surface effect of the first droplet on the sensor.

A second phase response 1152 is provided in which the capacitance decreases to a first characteristic capacitance C5. The second signal response corresponds to a bulk effect of the first droplet on the sensor. The time for the response, t5, is 7.00 seconds.

A second droplet engages 1143 with the sensor providing a second signal response. The second signal response includes a first phase response 1161 in which the capacitance increases almost instantaneously from around 2.5×10⁻⁸ F to 4.0×10⁻⁸ F. The signal to noise ration of the first phase response, s10, was measured as 3.31. The first phase response corresponds to a surface effect of the second droplet on the sensor.

A second phase response 1162 is provided in which the capacitance decreases to a second characteristic capacitance 010. The second phase response corresponds to a bulk effect of the first droplet on the sensor. The time for the response, t10, is 4.28 seconds.

The first and second droplets are removed 1145 from the sensor, providing a recovery signal response 1171. The time for the recovery signal response, td, is 12.00 seconds.

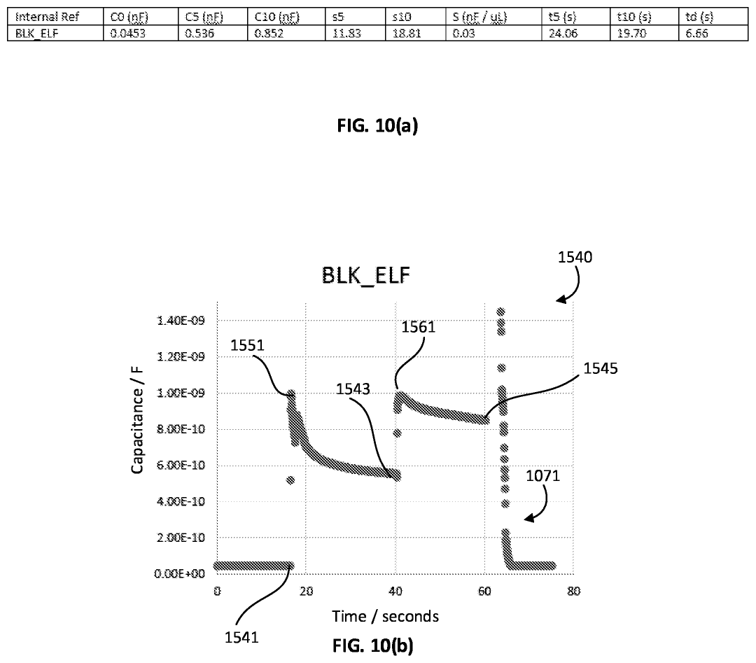

Referring now to FIG. 10, there is shown empirical data of a capacitance signal response measured by the two terminal device shown in FIG. 4 when incorporating sensor device with a substantially reduced groove aspect ratio. The sensor, identified as BLK_ELF is formed from a reference substrate including a series of grooves having a groove width of around 80 μm and a groove depth of less than 1 μm, corresponding to a groove aspect ratio of 0.013.

In common with the sensors described with respect to FIG. 6, the first and second faces of the grooves are formed with metallised faces providing the first and second electrically non-insulating portions formed as layers of aluminium approximately 100 nm thick.

The sensor BLK_ELF includes air as a detection medium. FIG. 10(b) shows the signal response 1540 of sensor BLK_ELF in response to a first and second droplet of analyte, in this case liquid water, as explained in reference to FIG. 4. The metrics of signal response are provided in the table shown in FIG. 10(a), corresponding the metrics of the model response explained with reference to FIG. 5.

A first droplet engages 1541 with the sensor providing a first signal response. The first signal response includes a first phase response 1551 in which the capacitance increases almost instantaneously from a baseline of around 0 F to around 1.0×10⁻⁹ F, a change of around 1.0×10⁻⁹ F, or 1 nF.

A second droplet engages 1543 with the sensor providing a second signal response. The second signal response includes a first phase response 1561 in which the capacitance increases almost instantaneously from around 6.0×10⁻¹⁹ F to around 1.0×10⁻⁹ F, a change of around 4.0×10⁻¹⁹ F, or 0.4 nF.

Thus, in comparison with the first phase responses 1051, 1061 of the BLK-IGI example shown in FIG. 6, the respective first phase responses 1551 and 1561 of BLK-ELF are substantially weaker. That is the change in capacitance of the BLK-IGI sample is of a magnitude, in the first signal response of over 25 times greater, and in the second signal response of around 200 time greater, than that of BLK-ELF. The inventors thereby conclude that due to its groove aspect ratio, the BLK-ELF sensor device would be inadequate to provide a reliable signal response.

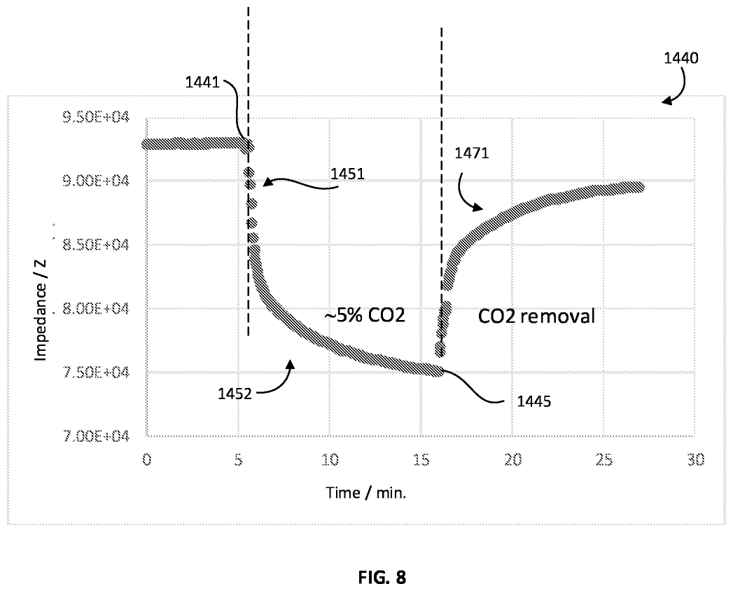

Referring now to FIG. 8 there is shown empirical data of a capacitance signal response of a further example embodiment electronic sensor device under test conditions in response to an atmosphere including elevated concentrations of carbon dioxide gas.

The sensor comprises the substantially the same groove configuration as the sensors of samples BLK_IGI and D53_1P described in reference to FIG. 6. The detection medium includes Dupont Luxprint 8155 mixed with an imidazolium ionic liquid (e.g. 1-ethyl-3-methylimidazolium bis(trifluoromethylsulfonyl)imide), the details of which are provided herein. Within the detection medium, the Dupont Luxprint 8155 provides a binder. The imidazolium ionic liquid provides an active component.

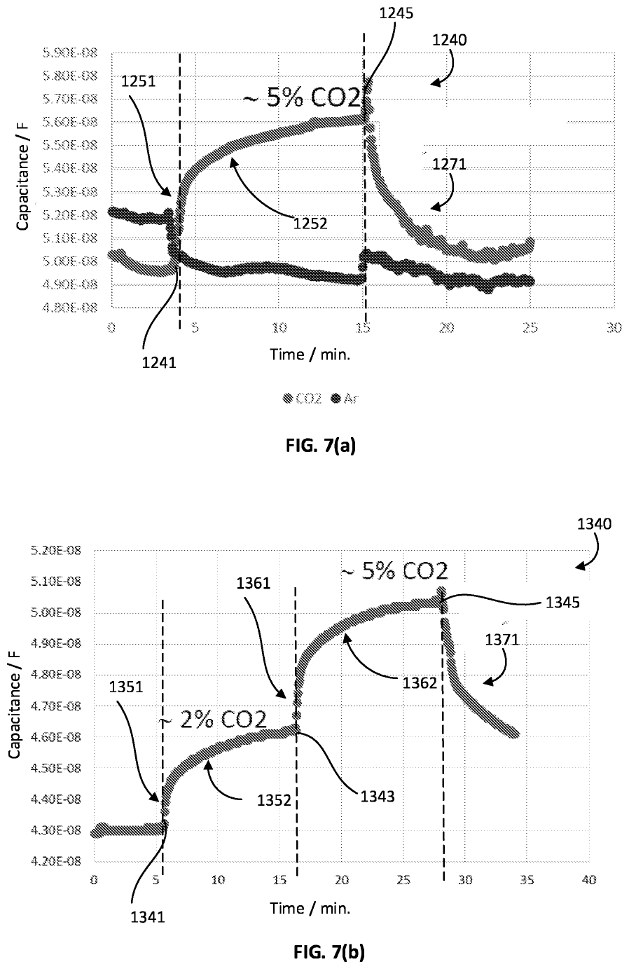

FIG. 7(a) shows the signal response 1240 of sensor in response to the introduction an analyte, in this case an atmosphere of 5% carbon dioxide, as explained with reference to FIG. 6. A control signal response including a sensor device to which an argon atmosphere is provided is also shown.

Carbon dioxide engages 1241 with the sensor providing a signal response. The signal response includes a first phase response 1251 in which the capacitance increases from a baseline of around 5.0×10⁻⁸ F to at least 5.4×10⁻⁸ F. The first phase response corresponds to a surface effect of the carbon dioxide on the sensor. The time for the first phase response to occur is around 1 minute.

A second phase response 1252 is provided in which the capacitance increases at a second, in this case slower, rate to a first characteristic capacitance of around 5.6×10−8 F. The phase signal response corresponds to a bulk effect of the carbon dioxide on the sensor. The time for the response is around 6 minutes.

The 5% carbon dioxide atmosphere is discontinued by removing 1245 the lid of a test chamber holding the sensor during testing. Discontinuation of the 5% carbon dioxide from the sensor provides a recovery signal response 1271.

FIG. 7(b) shows the signal response 1340 of sensor in response to the introduction an analyte, as explained with reference to FIG. 4. Specifically, in the example used to obtain the data of FIG. 7(b) the analyte is provide by an initial atmosphere of 2% carbon dioxide, followed by a subsequent atmosphere of 5% carbon dioxide.

With the initial carbon dioxide atmosphere, carbon dioxide engages 1341 with the sensor providing a first signal response. The first signal response includes a first phase response 1351 in which the capacitance increases from a baseline of around 4.3×10⁻⁸ F to at least 4.4×10⁻⁸ F. The first phase response corresponds to a surface effect of the carbon dioxide on the sensor. The time for the first phase response to occur is less than 1 minute.

A second phase response 1352 is provided in which the capacitance increases at a further, in this case slower, rate towards a first characteristic capacitance of around 4.6×10⁻⁸ F. The second phase response corresponds to a bulk effect of the carbon dioxide on the sensor. The time for the response is around 10 minutes.

With the introduction of the subsequent carbon dioxide atmosphere, carbon dioxide engages 1343 with the sensor providing a second signal response. The second signal response includes a first phase response 1361 in which the capacitance increases from around 4.6×10⁻⁸ F to at least 4.8×10⁻⁸ F. The first phase response corresponds to a surface effect of the increased concentration of carbon dioxide on the sensor. The time for the first phase response to occur is less than 1 minute.

A second phase response 1362 is provided in which the capacitance increases at a further, in this case slower, rate towards a second characteristic capacitance of around 5.0×10⁻⁸ F. The second phase response corresponds to a bulk effect of the increased concentration of carbon dioxide on the sensor. The time for the response is around 10 minutes.

The 5% carbon dioxide atmosphere is discontinued by removing 1345 the lid of a test chamber holding the sensor during testing. Discontinuation of the 5% carbon dioxide from the sensor provides a recovery signal response 1371.

Referring now to FIG. 8, there is shown empirical data of corresponding to measuring an impedance signal response during the introduction of an atmosphere including 5% carbon dioxide gas as described above in reference to FIG. 7(a). In this way, the corresponding impedance signal response is shown including substantially the same features as the capacitance signal response.

Carbon dioxide engages 1441 with the sensor providing a signal response. The signal response includes a first phase response 1451 in which the impedance decreases from a baseline of around 9.3×10⁴ Z to below 8.5.×10⁴ Z. The first phase response corresponds to a surface effect of the carbon dioxide on the sensor. The time for the first phase response to occur is less than 1 minute.

A second phase response 1452 is provided in which the impedance decreases at a second, in this case slower, rate to a first characteristic impedance of around 7.5×10⁴ Z. The second phase response corresponds to a bulk effect of the carbon dioxide on the sensor. The time for the response is around 9 minutes.

The 5% carbon dioxide atmosphere is discontinued by removing 1445 the lid of a test chamber holding the sensor during testing. Discontinuation of the 5% carbon dioxide from the sensor provides a recovery signal response 1471.

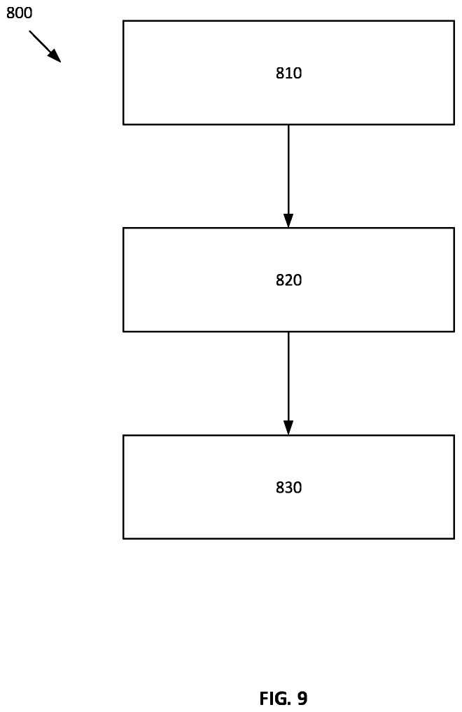

Referring now to FIG. 9, there is shown an example method 800 of forming an electronic sensor device according to an aspect of the invention.

The method 800 starts by providing 810 a substrate. In the next step 820, a groove is then formed within a surface of said substrate of the type described herein. Thus, the groove provides a first face, a second face and a groove profile including at least a groove depth within the substrate and a groove width at the surface of the substrate. The forming also provides the first face such that it includes a first electrically non-insulating portion and forms the second face such that it includes a second electrically non-insulating portion. The first electrically non-insulating portion is electrically separated from said second electrically non-insulating portion within the profile.

In the final step 830, a detection medium is provided within at least a portion of said profile so as to contactingly engage said first and second electrically non-insulating portions

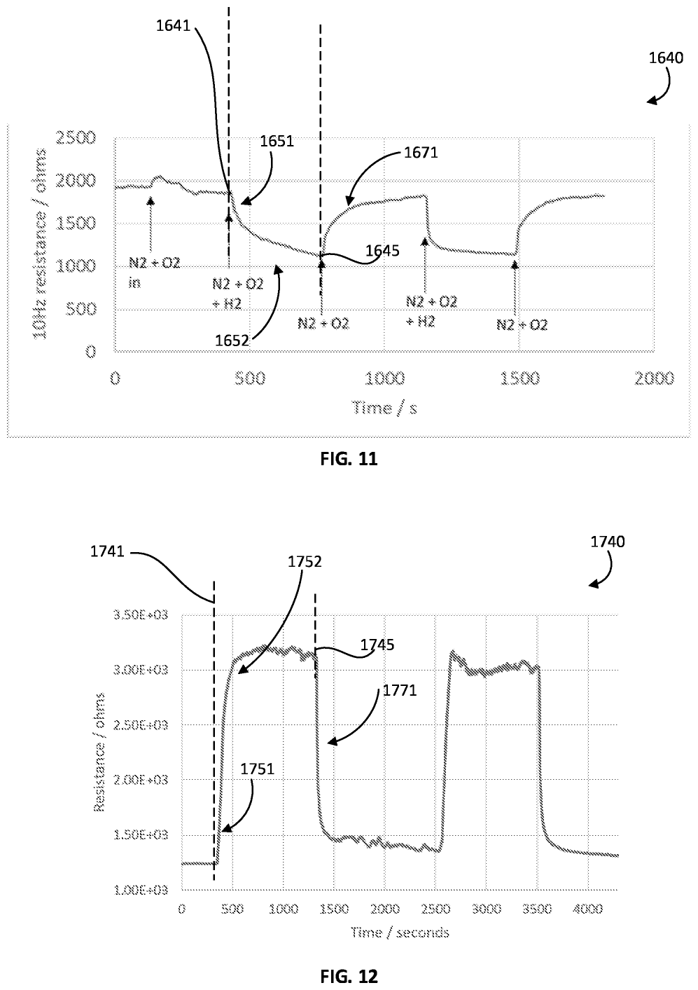

Referring now to FIG. 11, there is shown empirical data of corresponding to measuring a resistance signal response during the introduction of an atmosphere including 2% hydrogen gas into a baseline mixture of oxygen and nitrogen gases. In this way, the corresponding resistance signal response 1640 is shown including substantially the same features as the capacitance and impedance signal responses.

Hydrogen engages 1641 with the sensor providing a signal response. The signal response includes a first phase response 1651 in which the resistance decreases from a baseline of around 1900 ohms to at below 1600 ohms. The first phase response corresponds to a surface effect of the hydrogen on the sensor. The time for the first phase response to occur is less than 1 minute.

A second phase response 1652 is provided in which the resistance decreases at a second, in this case slower, rate to a first characteristic resistance of around 1150 ohms. The second phase response corresponds to a bulk effect of the hydrogen on the sensor. The time for the response is around 4 minutes.

The 5% hydrogen atmosphere is discontinued by introduction 1645 of an atmosphere of the baseline mixture of oxygen and nitrogen gases during testing. Discontinuation of the 5% hydrogen from the sensor provides a recovery signal response 1671.

Referring now to FIG. 12, there is shown empirical data of corresponding to measuring a resistance signal response 1740 during the introduction of an atmosphere including 2% hydrogen. In this way, the corresponding resistance signal response is shown.

Hydrogen engages 1741 with the sensor providing a signal response. The signal response includes a first phase response 1751 in which the resistance increases from a baseline of around 1240 ohms to at below 1600 ohms. The first phase response corresponds to a surface effect of the hydrogen on the sensor. The time for the first phase response to occur is approximately 30 seconds.

A second phase response 1752 is provided in which the resistance increases at a second, in this case slower, rate to a first characteristic resistance of around 3200 ohms. The second phase response corresponds to a bulk effect of the hydrogen on the sensor. The time for the response is around 2 minutes.

The 2% hydrogen atmosphere is discontinued by introduction 1745 of an atmosphere of the baseline mixture of oxygen and nitrogen gases during testing. Discontinuation of the 2% hydrogen from the sensor provides a recovery signal response 1771.

It will be appreciated by persons skilled in the art that the above detailed examples have been described by way of example only and not in any limitative sense, and that various alterations and modifications are possible without departing from the scope of the invention as defined by the appended claims. Various modifications to the detailed examples described above are possible, for example, variations may exist in the number, shape, size, arrangement, assembly or the like of grooves, groove faces and groove cross-sectional profiles. Various modifications may also exist in the nature and fill level of the detection media in order to tune the signal response of a sensor device.

1. An electronic sensor device comprising:
a substrate comprising at least one groove, said groove including a first face and a second face, said groove having a cross-sectional profile including at least a groove depth within said substrate and a groove width at a surface of said substrate;
said first face including a first electrically non-insulating portion and said second face including a second electrically non-insulating portion wherein, within said profile, said first electrically non-insulating portion is electrically separated from said second electrically non-insulating portion; and
a detection medium provided within said groove, arranged to contactingly engage said first and second electrically non-insulating portions, and adapted to be contactingly engaged by an analyte;
wherein said profile or said detection medium is adapted such that, in use, a signal response is provided comprising a first response phase and a subsequent second response phase in response to an engagement of the analyte with said detection medium.

2. An electronic sensor according to claim 1, wherein said detection medium comprises a medium surface and a medium body, and wherein said first response phase is provided in response to an engagement of the analyte with said medium surface.

3. An electronic sensor according to claim 2, wherein said second response phase is provided in response to an engagement of the analyte within said medium body.

4. An electronic sensor according to claim 2, wherein said medium body comprises a portion of said substrate forming said profile of said at least one groove.

5. An electronic sensor according to claim 4, wherein said profile of said at least one groove has a first characteristic shape and wherein, in use, in second response phase said profile deforms to a second characteristic shape.

6. An electronic sensor according to claim 1, wherein said signal response comprises a change in at least one predetermined electrical characteristic of said at least one groove.

7. (canceled)

8. An electronic sensor according to claim 6, wherein, within said first response phase, said predetermined electrical characteristic changes at a first rate and, within said second response phase, said predetermined electrical characteristic changes at a second rate.

9. (canceled)

10. An electronic sensor according to claim 8, wherein said first response phase lasts for a first time interval and said second response phase lasts for a second time interval.

11-12. (canceled)

13. An electronic sensor device according to claim 1, wherein said groove width is less than or equal to 100 μm.

14. An electronic sensor device according to claim 1, wherein a groove aspect ratio is defined as the ratio of said groove depth to said groove width, and wherein said groove aspect ratio is within the range 0.1:1 to 50:1.

15. An electronic sensor device according to claim 1, wherein said profile is any one of: U-shaped, V-shaped, asymmetrically V-shaped, rounded, semi-circular, or square shaped.

16. (canceled)

17. An electronic sensor device according to claim 1, wherein said first or second electrically non-insulating portion extends from said respective first or second face onto said surface of said substrate.

18. An electronic sensor device according to claim 1, wherein said first or second electrically non-insulating portion comprises a coating.

19. An electronic sensor device according to claim 1, wherein said at least one groove comprises a series of grooves, and wherein a second face of a first groove of the said series of grooves is in electrical communication with a first face of a second groove of said series of grooves.

20-23. (canceled)

24. An electronic sensor device according to claim 1, wherein said detection medium is conformally deposited onto at least a portion of said profile.

25. (canceled)

26. An electronic sensor device according to claim 1, wherein said detection medium comprises a binder and an active component in a mixed state.

27. (canceled)

28. An electronic sensor device according to claim 26, wherein said signal response is provided in response to an engagement of the analyte with said active component.

29. An electronic sensor device according to claim 26, wherein said active component is provided as a solid filler or as a liquid.

30. An electronic sensor device according to claim 26, wherein said binder is adapted to bind said active component within said detection medium.

31-33. (canceled)

34. An analyte detection apparatus comprising:
an electronic sensor according to claim 1, and
a detector,
wherein said first electrically non-insulating portion of said at least one groove forms a first electrode and said second electrically non-insulating portion of said at least one groove forms a second electrode, and
wherein said first and second electrodes are electrically connected to said detector such that said detector detects and measures said signal response.

35-37. (canceled)
